(19) United States
(12) Patent Application Publication
(10) Pub. No.: US 2013/0004993 A1
(43) Pub. Date: Jan. 3, 2013

(54) Green Chemical Process to Produce Natural Products

(75) Inventor: Thomas J. Manning, Hahira, GA (US)

(73) Assignees: University System of Georgia, Atlanta, GA (US); Valdosta State University, Valdosta, GA (US)

(21) Appl. No.: 13/170,855

(22) Filed: Jun. 28, 2011

(51) Int. Cl. C12P 21/00; C12N 1/26; C12N 1/14; C12N 1/12; C07D 305/14; C12P 17/18; C07D 515/22; C07K 1/14; C07D 493/22; C12P 17/02; C12N 1/20; C12N 1/22

(52) U.S. Cl. 435/71.2; 435/253.6; 435/248; 435/256.8; 435/257.1; 435/252; 435/119; 540/466; 435/71.1; 530/344; 549/344; 530/321; 549/267; 435/123; 549/510

(57) ABSTRACT

Methods for producing and obtaining natural products from microbial amplification chambers are described. This approach utilizes the concept of green chemistry to synthesize and extract the marine and terrestrial natural products. The method describes techniques to colonize and grow the selected bacteria and to continuously harvest the pharmaceutical agent from the broth without using any commercial solvents.

The inventor of this patent application is not an employee of the United States Government.

### BACKGROUND OF THE INVENTION

1. Field of Invention

The present invention relates to a method to produce and extract marine natural products. It adheres to the twelve principles of a green technology published by the environmental protection agency. In this demonstration, the process produces bryostatin from a microbial amplification chamber, which allow specific microbes to colonize and multiply, that primarily reside in a marine environment. The bryostatins are known pharmaceutical agents that are being tested for medical effects on cancer and Alzheimer's patients.

2. Description of Related Art

Natural products are medicinal agents that are extracted from organisms. They have been found in marine and terrestrial organisms. Many natural products are difficult or impossible to produce using current laboratory methods such as organic synthesis or genetically modified microbes. Hence, a more effective, universal technique to produce natural products that follows the principals of green chemistry is described in this application. Our novel method is demonstrated using the marine natural product bryostatin.

Since the 1980's, compounds containing bryophan rings were discovered and tested for medicinal properties. They shared a common ring and other structural features but have one or two side groups that vary (i.e. —H, ‘3OH, —Ac). To date there have been twenty structures of bryostatin isolated and identified. Bryostatin-1 was originally demonstrated to be effective against leukemia and has since been studied in numerous cell line and human trials. U.S. Pat. No. 4,560,774 disclosed the structures of bryostatins-1, 2 and 3. U.S. Pat. No. 4,611,066 was granted for the structures of five bryostatins (4 though 8). U.S. Pat. No. 4,833,257 was granted for the structures of bryostatins 9 to 13. U.S. Pat. No. 5,072,004 focused on the synthetic conversion of bryostatin-2 to bryostatin-1. U.S. Pat. No. 6,900,339 revealed a discovery in which bryostatin was harvested in high yields from the larvae of Bugula neritina and revealed the structure of bryostatin-20. U.S. Pat. No. 6,825,229 is a method for treating Alzheimer's disease that uses bryostatin-1. U.S. Pat. No. 7,045,495 combines bryostatin and paclitaxel for treating cancer. The last two patents mentioned here are used as representative of bryostatins importance in medicinal applications. Its limited supply dramatically limits its medical uses.

Over the past two decades green chemistry has gained a foothold in both industrial and academic labs. Green methods of synthesis focus on safer methodologies that have little or no impact on the environment. The role of various aspects of chemical synthesis such as the expanded role of catalysis has been a focus of green processes for some time. In addition to safety, the concept of sustainable chemical processes that do not hurt the planet is a fundamental premise of the green approach. The solvent selection for green chemistry is important from several perspectives including safety, recycling and solubility of the compound. Currently green chemistry is still described as an emerging field. While it may be difficult, if not impossible for some chemical processes to hit all twelve principles described by the United States Environmental Protection Agency, it is reasonable for every chemical synthesis to achieve some of the principles.

Marine natural products have risen in profile as pharmaceutical agents over the past thirty years. Sources of medicinal agents from the world's oceans have included bacteria, corals, sea squirts, bryozoans, and algae. Marine natural products extracted from these organisms are usually found in extremely low concentrations with values in the 10⁻⁵ to 10⁻⁷% range, by mass, being typical. There is a tremendous diversity of life in the marine environment, particularly at the microbial level. Many of these microbes, from those dwelling in water columns to species residing in deep ocean sediments and those enjoying symbiotic relationships with other marine organisms, have yet to be discovered.

While a host of marine life resides in the worlds waters, the invertebrates are among the most abundant and the most overlooked. The bryozoans Bugula neritina is one of the more studied invertebrates found not only along the coast of Florida, but other regions of North America, Asia and Europe. Bryostatin is a marine natural product that has at least twenty structural variations but this number is likely to increase as many esterfication reactions involving the R1 and R2 group and chemicals found in nature are possible. It is extracted from the bryozoa Bugula neritina. Bugula is a sessile invertebrate that is found, on a seasonal basis, at a range of locations in temperate waters. Bugula is a millimeter sized filter feeder that forms colonies that are typically in the 5-20 cm length range. In the Northern Gulf of Mexico the colonies first appear in late January or early February and are typically gone by early June. Bugula was part of the first large scale survey of marine organisms for medicinal agents. That study was initiated by the National Cancer Institute and the Bugula collections were handled by the Gulf Specimen Marine Lab in Panacea (Florida). The structure of Bryostatin-1 was first solved by George Pettit's research group located at Arizona State University. Bryostatin-1 has been tested in numerous cell line and human clinical trials over the past thirty years and has demonstrated strong pharmaceutical activity. It is now being studied as a cure of Alzheimer's disease. It is recognized within the scientific community that bryostatin is produced by a symbiotic bacteria that resides within or on Bugula but no evidence exists that a single species of bacteria is bryostatins sole source.

The first total organic synthesis of any bryostatin was that of bryostatin-7 published in 1990. Since other total synthesis have been reported including that of bryostatin-16, which incorporated a catalysis that helped reduce the number of steps used in any total synthesis of bryostatins. At the present time all synthesis of bryostatins require dozen's of steps, make extensive use of toxic solvents, produce waste, and make these approaches a difficult commercial endeavor and beyond the scope of a green process. Currently, the total synthesis methodologies are viewed as having no commercial value due to their complexity and cost.

Other research related to bryostatin production has focused on the genetic manipulation of marine bacteria. Marine bacteria are often difficult to grow in a laboratory setting. Past attempts have been made to remove specific genes from bacteria believed to produce bryostatin and relocate this genetic material to bacteria that are easier to grow in a laboratory setting. These efforts have yet to produce bryostatin in any quantity.

Likewise, efforts have been made to grow large quantities of Bugula in the marine environment (Pacific Ocean), harvest the organism and extract the bryostatins. These large scale commercial efforts have also meet with limited success and subsequently not considered an economical source. In reviewing organic synthesis, genetics of marine bacteria and large scale aquaculture of the host organism, none of these approaches have yielded commercial quantities of bryostatin. Currently bryostatin's are still harvested from Bugula neritina and extracted with very low yields (10⁻⁶ to 10⁻⁷% being a typical quantity). Bugula is a seasonal species that can be difficult to find from year to year. In its harvest, the amount of by-catch that is destroyed can be tremendous. A method is described in this application that describes a green process for the production of bryostatins. This invention has also been applied to other marine and terrestrial natural products. Research by the inventor has shown that the medicinal agents taxol and ET743 can be produced using the methodology described in this document with some alteration to the exact substrate composition.

### BRIEF SUMMARY OF DISCLOSURE

An object of the invention is to overcome the drawbacks relating to the compromise designs of prior art devices as discussed above. The bryostatins have well known medicinal properties against diseases such as cancer and Alzheimer's. The bryostatins are difficult to produce synthetically, genetic manipulation of marine bacteria that produce bryostatin have not been successful, and harvesting bryostatins from Bugula results in an extremely low yield.

The method described here is a form of marine aquaculture where a marine bacterium, which is normally symbiotic with Bugula neritina, is grown in a marine environment. Once they have colonized a respective surface and material, they are moved to a secondary containment unit where the bryostatin is harvested. While it is possible to solvent extract bryostatins from the materials and nutrients used to grow the bacteria, the solvents that are involved in these steps can have detrimental effects on workers and the environment. This application reveals how cotton, treated with a basic solution, is used to extract bryostatin from a marine ecosystem that has high bacteria levels.

The method described in this application develops a technique called microbial amplification chambers that are used to colonize and provide nutrients to microbial species in a marine environment. Once a microbial colony is well established, it is moved to a controlled containment unit such as a runway. At this point if it is harvested for bryostatin using traditional solvent methods (i.e. solvent extraction with dichloromethane) than the support material that maintains the colonies are destroyed. Unbleached cotton is reacted with a basic solution (pH>8) and is rinsed with water. This reacted natural polymer is used to selectively absorb the bryostatins from solution. Unreacted cotton or some other natural polymers (i.e. different forms of cellulose, lignin, chitin) also absorbed bryostatins but in lower quantities. While cotton will absorb a host of chemicals from solution, it does serve to concentrate molecular species, such as bryostatin, that has limited solubility in water. A simple analogy is the absorption of organic dyes in cotton as carried out by the clothing industry on a wide scale.

Placing the natural polymer in the runway for an extended period of time (i.e. days) allows the bryostatins to adsorb onto the material. This does not remove all bryostatins in a single extraction but, as a decided advantage verses solvent extraction, it does not destroy the microbial communities. The second advantage of using cotton to remove bryostatin from a complex broth that contains many chemicals and living species is that it conforms to the principals of green chemistry. It does not use common solvents such as carbon tetrachloride, dichloromethane, benzene, toluene, acetone or acetonitrile, which can present health, safety and disposal problems. This allows for the ongoing production of bryostatins isolated at a consistent rate from a laboratory. In using a 12 feet by 3 feet piece of cotton and leaving it in a fifteen foot long (2 feet deep, 2 feet wide) runway for one week allowed for the extraction of a raw product that weighed five grams. It contained a mix of bryostatins.

### DETAILED DESCRIPTION OF THE DISCLOSURE

In this application a new approach to synthesizing marine natural products is outlined. There is a particular focus on the green aspects of the process, particularly in the synthesis stage. As opposed to organic synthesis, genetics or aquaculture of the bryozoa, our approach focuses on providing a physically, chemically and biologically friendly environment for the symbiotic bacteria that produces bryostatin to colonize and thrive. Particularly in the early stages of colonization, our process acknowledges that marine bacteria are extremely difficult or impossible to grow in a lab setting and some colonization should start in the marine environment meaning a healthy colony under the correct conditions is transferred to the substrate described in this work. Research resulting in this invention was conducted by the inventor over the past decade and is described in detail in the listed publications. This work has established that bryostatin is not only found in Bugula neritina, as previously believed by the scientific community, but was also found in a number of other marine organisms in the same ecosystem as well as the marine sediment. This resulted in a scientific model being developed by the inventor that explained how marine bacteria, originally in marine sediment, become suspended in the water column and subsequently colonized surfaces, substrates or organism that will chemically, physically and biologically support the bacteria colony. The ultimate goal of this research, which is revealed in this invention, was to provide a substrate favorable for microbial colonization and bryostatin production. Electron microscope studies indicate that many species of bacteria are present but there is no method available to easily determine if one or all of the species, perhaps numbering in the hundreds, produce bryostatins.

This process consists of several steps that include:

1. Nutrients. In this process dozens of chemicals are coated or soaked on a surface and the material is placed in a container. The substrate is used to colonize and feed the bacteria in the field. The nutrients range from sugars and proteins to nitrates and phosphates. Table 2 provides a sample nutrient mix used to produce a stable colony of bacteria that continuously produce bryostatins over several months. In this case, 454 grams of sand from the Bugula ecosystem is used as the substrate. Additional chemicals are added and the mixture is allowed to stabilize in the host ecosystem in a specific location in the Gulf of Mexico. The nutrient laden substrate, colonized by marine bacteria from a specific ecosystem, is returned to the lab and remains stable for several weeks, producing bryostatins.

2. Site selection. A large part of our research program over the past decade has focused on selecting the site for the grow outs and understanding its chemistry. This has included work in the marine environment for bryostatin and ET743 and the terrestrial environmental for taxol. Changing locations for the substrate deployment or sand collection by a few meters can change yields. This ongoing project has shown that different sites have a specific chemical signature, particularly the sediment. Using techniques such as inductively coupled plasma-atomic emission spectrometer (ICP-AES), Liquid Chromatography-Mass Spectrometry (LC-MS), Matrix Assisted Laser Desorption Ionization-Time of Flight Mass Spectrometry (MALDI-TOF-MS), Fourier Transform Infrared Spectroscopy (FT-IR), Ultraviolet-Visible Absorbance Spectroscopy (UV/Vis) and Fourier Transform-Ion Cyclotron Resonance (FT-ICR) spectrometry we have measured dozens of elemental (i.e. Fe, Mn, Sr, Ca) concentrations and hundreds of molecular concentrations in specific sediment samples and used this data to determine our substrate composition.

3. Colonization Surface. The chemicals are absorbed and mixed with specific substrate. Surfaces tested have included natural sponge (dead), paper products, shredded plastic bottles, chips and saw dust from various types of trees, chitin sources such as shrimp shells, sand, limestone dust, and manmade materials such as metals, plastics and styrofoam.

4. Colonization time. We found that our grow outs only work in specific ecosystems and require a minimum duration for the bacterial colonies to grow. Rarely have we been able to collect a small water sample, return it to the lab, and extract out any detectable quantities of bryostatins. It is important that the substrate and chemicals reach a chemical, physical and biological equilibrium that is similar to the host ecosystem allowing the microbes to colonize and produces bryostatins.

5. Ecosystem to Runway. Providing nutrients to the substrate in the marine environment has produced bryostatins but the productivity of the substrate decreases as the nutrients and minerals dissipate. Our production level increases in some cases after surfaces/materials are moved to a seawater fed runway. A system like a runway feed with sea water allows us to control and monitor parameters, such as water quality, nutrient levels and natural product production, over a period of time.

6. Extraction and Purification. Using the mass of the nutrients added as the starting weigh, we have produced 0.014% mixed bryostatins (% yield by mass), which is well above the 10⁻⁷% that comes from Bugula neritina. This small scale grows out involved the extraction of all material, killing the bacterial colony. Working towards a green process, we sought a more sustainable and environmentally friendly method to extract the marine natural product. Cotton is immersed the broth and allow it to remain there for a period of time that can vary from minutes to days. Longer durations result in higher yields. In long term submersions by the cotton, bacteria colonies producing bryostatin will grow on the cotton. Natural products are organic in nature and often have some low polarity characteristics, allowing them to absorb onto the cotton. While this method does not result in a 100% yield over a period of time, it does provide a method to continuously remove molecular species without damaging the microbial colony.

Our approach involves low cost chemicals that are safe to use and involve little disposal (most are consumed in the grow out). The microbial amplification chambers utilize a substrate such as sand, limestone, metal, plastics, paper products, natural or manmade sponges and/or wood products as a medium for the microbes to colonize and flourish. In some cases this material is consumed (i.e. paper, saw dust) by the microbes. The substrate and nutrients (table 2) are immersed in a solution for 24-48 hours before being placed in the desired ecosystem. This matrix is placed in a perforated container for at least one week. Typically colder months require longer colonization times which can last two to three months. There are seven groups of chemicals and materials that are potentially added to MAC's and include.

1. Inorganic salts, both soluble and insoluble, that play a nutrient role (i.e. Fe⁺³, NO₃⁻, NH₄⁺, S⁻², PO₄⁻³, etc.) are included.

2. Hydrocarbon species common in marine sediment such a different carboxylic acids, alcohols, squalene and humic/humus substances.

3. The organic nutrients included in material amplification chambers include vitamins, amino acids, peptides, protein starch and sugars. Some of these may use natural sources. For example, fresh potato slices are used as a source of starch.

4. The chambers include naturally occurring polymers such as proteins, peptides, cellulose, chitin, and lignin. Chitin has come from shrimp and crab shells while protein sources have included peptone, meats and commercially available protein supplements. Cellulose has come from man-made sponges while different forms of wood (chips, dust, etc) have served as sources of cellulose and lignin.

5. Inorganic species that help define the local ecosystem are used and can comprise a high percentage of the total mass. Species such as sodium silicate, calcium carbonate, calcium sulfate, and sand have been added as powders and/or rocks to the matrixes. The carbonates will release (slowly) small amounts of CO₂ in the anaerobic environment providing an essential gas. The decomposition of carbonates to carbon dioxide provides a natural pH buffer.

6. In some cases a host organism has been added to the matrix. For example, Bugula has been added to introduce the correct microbes and provide limiting nutrients that may not be present elsewhere. This is not done on a consistent basis and the presence of Bugula is not considered essential or necessary for the success of the colony if the bucket is placed in the correct location.

7. The last group added can include one or more man-made materials such as different metals and alloys, Styrofoam and other perforated plastics, graphite rods, paper products, plastics and glasses. These have produced bryostatins at different rates but typically hard, nondegradable, impenetrable surfaces are the least productive substrates. Some surfaces, such as metals and alloys, may provide nutrients and degrade over time.

Table 2 provides data for a specific mixture. In this case sand from the host ecosystem is used as a source of bacteria and as the substrate. In addition to water, other chemicals are added to provide a chemical environment that allows the microbes to thrive. There is no attempt to selectively grow one specific species of bacteria as no real evidence exists to definitely show only one species of bacteria produces bryostatin. This process adheres closely to the principles of green chemistry. The twelve principles of a green technology and how this process adheres to that chemical process are defined below:

1. Prevent waste: No commercially produced solvents are used in the production or isolation of marine natural products using this process.

2. Synthetic methods incorporate materials into the final product: Our materials are either re-used or consumed by the microbes. Nutrients such as sugars, phosphates, nitrates and amino acids are periodically replenished after being consumed while support materials such as limestone, sand and sponges are re-used.

3. Synthetic methods generate substances that possess little or no toxicity: This process is producing marine and terrestrial natural products. Once a microbial grow out is complete, only sponges, sand, minerals, cotton etc. remain. They are reused until they degrade or are consumed.

4. Chemical products should preserve function: Our natural products are produced by the same organisms that were the original synthetic sources. By using cotton to remove the medicinal agents from the matrix, all chemical and biological functions remain intact.

5. Chemical materials (e.g. solvents, separation materials) should be minimized: Bacteria functioning in a marine environment produce our drugs. Items commonly used in organic synthesis such as common solvents (i.e. aromatic, chlorinated), and chromatography columns are not used in this process.

6. Energy requirements be minimized: Our grow outs mimics an aquaculture enterprise. There is little/no electricity used in this outdoor pharmaceutical aquaculture invention.

7. A raw material or reactants should be renewable: We have focused on buying materials for our broths that are inexpensive and are produced by multiple sources.

8. Reduce derivatives: This process requires no derivatives.

9. Catalytic reagents are better than a stoichiometric reactant: When nutrients are added to a substrate, all consumables (sugars, amino acids, phosphates, etc.) are restocked on a regular basis.

10. Chemical products should break into environmental friendly species: Our nutrients are essentially consumed by the microbes and the remnants are materials taken directly from nature (limestone, sponge, wood chips, etc).

11. Analytical methodologies should be real-time: We have been working on a TLC method to detect bryostatins in the field but found matrix effects can interfere extensively.

12. Species used should be chosen to minimize accidents: Although any chemical can cause problems under the wrong circumstances, many of our reactants are handled by the general public on a daily basis in a safe manner.

In addition to the EPA's Twelve Principles of Green Chemistry, this inventions takes into account the economics of natural product production on a large scale, the ability to adapt the process for different natural products produced in different locations, and the ability to serve as a process to discover new natural products. For example, our colonization approach can be applied to deep ocean environments where pressures, temperatures and the exact chemical and physical conditions can be difficult to produce in a laboratory setting. Currently Bugula neritina is the primary source of bryostatin, which requires large scale harvesting of the marine organism which can result in environmental destruction and massive loss of sea life in by-catch.

The Nuclear Magnetic Resonance (NMR, 500 MHz) data obtained in our analysis utilized proton (¹H) and carbon (¹³C) spectra, in one and two (COSY, HETCORR, DEPT) dimensional experiments. This data was compared to NMR spectral data published for the bryostatin in the peer reviewed scientific literature. The correlation coefficients between our data and the accepted values are excellent for both data sets (r²>0.99). Techniques such as ultraviolet-visible absorbance spectrometer (UV/Vis), Fourier transform infrared spectrometry (FT-IR), Matrix Assisted Laser Desorption Ionization Time of Flight Mass Spectrometer (MALDI-TOF-MS), Liquid Chromatography-Mass Spectrometry (LC-MS), and ¹H, ¹³C and ¹⁵N Nuclear Magnetic Resonance (NMR) were used throughout this project to qualitatively and quantitatively identify bryostatin structures and molecular fragments.

TABLE 1
The mass of the different bryostatins and the bryostatins plus
sodium. Exact masses and fragmentation patterns are used in
mass spectrometric measurements to identify bryostatins.

| | | M.M + | |
|---|---|---|---|
| | Monoisotopic | (Na+) | Empirical |
| Bryostatin # | Mass | 22.9892 | Formula |
| 1 | 904.4456 | 927.4348 | C₄₇H₆₈O₁₇ |
| 2 | 862.4350 | 885.4243 | C₄₅H₆₆O₁₆ |
| 3 | 888.4143 | 911.4035 | C₄₆H₆₄O₁₇ |
| 4 | 894.4613 | 917.4505 | C₄₆H₇₀O₁₇ |
| 5 | 866.4300 | 889.4192 | C₄₄H₆₆O₁₇ |
| 6 | 852.4143 | 875.4035 | C₄₃H₆₄O₁₇ |
| 7 | 824.3830 | 847.3722 | C₄₁H₆₀O₁₇ |
| 8 | 880.4456 | 903.4348 | C₄₅H₆₈O₁₇ |
| 9 | 852.4143 | 875.4035 | C₄₃H₆₄O₁₇ |
| 10 | 808.4245 | 831.4137 | C₄₂H₆₄O₁₅ |
| 11 | 766.3775 | 789.3667 | C₃₉H₅₈O₁₅ |
| 12 | 932.4769 | 955.4661 | C₄₉H₇₂O₁₇ |
| 13 | 794.4088 | 817.3980 | C₄₁H₆₂O₁₅ |
| 14 | 824.4194 | 847.4086 | C₄₂H₆₄O₁₆ |
| 15 | 920.4405 | 943.4297 | C₄₇H₆₈O₁₈ |
| 16 | 790.4139 | 813.4031 | C₄₂H₆₂O₁₄ |
| 17 | 790.4139 | 813.4031 | C₄₂H₆₂O₁₄ |
| 18 | 808.4245 | 831.4137 | C₄₂H₆₄O₁₅ |

TABLE 2
Sample of chemical mixture used to maintain a bacterial
ecosystem that produces bryostatins. The sand is obtained
from the host ecosystem and is the source of the bacteria
as well as the primary substrate.

| | mass |
|---|---|
| Species | (grams) |
| Water (RO) | 1000 |
| Sand (host ecosystem) | 454 |
| Chloride ion | 19.29 |
| Sodium ion | 10.78 |
| calcium carbonate | 5 |
| sodium silicate | 5 |
| Na₂NTA | 3.0 |
| iron(III)chloride | 2.5 |
| Sulfate ion | 2.6 |
| Magnesium ion | 1.5 |
| wood dust (cellulose/lignin) | 1 |
| potato (starch) | 1 |
| Steel wool | 1 |
| calcium chloride | 0.5 |
| Potassium ion | 0.42 |
| Glucose | 0.4 |
| peptone | 0.25 |
| Bicarbonate ion | 0.21 |
| sodium succinate | 0.15 |
| Yeast | 0.1 |
| Chitin | 0.1 |
| Sodium nitrate | 0.075 |
| Bromide ion | 0.056 |
| Sodium acetate | 0.05 |
| Boric Acid | 0.0257 |
| Zinc sulfate hydrate | 0.022 |
| Sodium phosphate | 0.01 |
| Cobalt chloride hydrate | 0.01 |
| Strontium chloride hydrate | 0.01 |
| Potassium molybdenum hydrate | 0.0063 |
| Folic Acid (B9) | 0.002 |
| Fluoride ion | 0.001 |
| Vitamin C | 0.0007 |
| Thiamin (B1) | 0.0007 |
| Riboflavin (B2) | 0.0005 |
| Niacin (B3) | 0.0005 |
| Vitamin B6 | 0.0005 |
| Pantothenic acid (B5) | 0.0005 |
| Vitamin E | 0.0002 |
| Copper ion | 0.00002 |
| Vitamin A | 0.00001 |
| Folate | 0.000002 |
| Iodine | 0.0000015 |
| Chromium ion | 0.0000012 |
| Vitamin B12 | 0.0000006 |
| Biotin (B7) | 0.000001 |
| Vitamin K | 0.00000025 |
| Selenium ion | 0.0000002 |
| Vitamin D | 0.0000001 |

We claim:

1. A method of producing marine natural products using the principles of green technology by immersing a substrate in a marine environment.

2. A method of preparing a substrate with nutrients that results in a proper colonization environment for microbes.

3. The method of claim 2, wherein one of the groups of nutrients are sugars.

4. The method of claim 2, wherein one of the groups of nutrients is hydrocarbons or functionalized hydrocarbons.

5. The methods of claim 2, wherein one of the groups of nutrients is proteins, peptides and amino acids.

6. The methods of claim 2, wherein one of the groups of nutrients is composed of anions and cations that combine to form soluble species such but not limited to salts of phosphates, nitrates, chlorides, bromides, iodides, sulfates, sulfides, sodium, potassium, calcium, magnesium, iron, and zinc.

7. The methods of claim 2, wherein one of the groups of nutrients is composed of anions and cations that combine to form insoluble or slightly soluble salt species such but not limited to carbonates, oxides, hydroxides, sulfates, carboxylates, aminocarboxylates, vanadium, iron, chromium, and calcium.

8. The methods of claim 2, wherein one of the groups of materials is metals in their elemental form such as iron, copper and zinc or alloys based on these elements that include steel.

9. The methods of claim 2, wherein one of the groups of materials is organic based nutrients such as vitamins.

10. The methods of claim 2, wherein one of the groups of materials is naturally occurring minerals such as sand or limestone.

11. The methods of claim 2, wherein one of the groups of nutrients is composed of natural polymers such as chitin, cellulose, starch and lignin.

12. The methods of claim 3 wherein these materials may be in the form of a purified chemical or a natural material such as but not limited to wood dust, vegetables, shrimp shells, cotton and marine sponges.

13. The method of claim 1, wherein the materials are held in a perforated container.

14. The method of claim 1, where the container can be removed from the environment, the host ecosystem, and the contents transferred to a runway where the microbes are placed in a static environment.

15. A method of removing the natural product from the broth that involves adsorption and bonding to a material such as cotton.

16. The method of claims 1 through 15 where no commercial solvent is used and the principles of green chemistry are adhered to throughout the process of removing the natural product using a natural polymer.

17. The methods of claims 1 through 16 are used to produce any marine natural product that is produced by a microbe such as but not limited to ET743, dolastatin, halichondrin, aplidine, spongastatin and bryostatin.

18. The methods of claims 1 through 16 are used to produce the marine natural product bryostatin.

19. The methods of claims 1 through 16 are used to produce the terrestrial natural products produced by organisms such as fungi, algae and bacteria.

20. The methods of claims 1 through 16 are used to produce the terrestrial natural product taxol.
